(19) United States
(12) Patent Application Publication
(10) Pub. No.: US 2009/0076520 A1
(43) Pub. Date: Mar. 19, 2009

(54) Apparatus For Treating Bone

(76) Inventor: Gil-Woon CHOI, Anyang-si (KR)

Correspondence Address: Edwards Angell Palmer & Dodge LLP, P.O. Box 55874, Boston, MA, 02205, US

(21) Appl. No.: 12/209,438

(22) Filed: Sep. 12, 2008

(30) Foreign Application Priority Data
Sep. 14, 2007 (KR) 10-2007-0093572

(51) Int. Cl. A61F 11/00; A61B 17/00; A61F 5/00

(52) U.S. Cl. 606/108; 606/79; 606/87

(57) ABSTRACT

An apparatus for treating a bone, which provides a path from the skin to a bone, is provided which comprises a cannula with an improved coupling structure, an expander, a needle, and a spacer. With the apparatus, operation time can be reduced. The cannula and the expander can be easily combined together or separated from each other. Further, the needle can be easily removed from the bone. In addition, an insertion space for a cavity-forming instrument, such as a balloon catheter, can be easily obtained inside the bone.

### CROSS-REFERENCE TO RELATED APPLICATION

This application claims under U.S.C. §119(a) on Korean Patent Application No. 10-2007-0093572 filed on Sep. 14, 2007, the entire contents of which are incorporated herein by reference.

### BACKGROUND

(a) Technical Field

The present disclosure relates to an apparatus comprising a catheter and an expander for treating a bone that provides a path from a skin to the bone.

(b) Background Art

Vertebroplasty is a medical procedure where the original height and angle of kyphosis of a fractured vertebra are restored, followed by its stabilization using injected bone filler material. The procedure is commonly done percutaneously. Height and angle restorations are carried out by using either hydraulic or mechanical intravertebral expansion.

Restorators and accessories are known as apparatuses used for vertebroplasty. A cavity-forming instrument, such as a balloon catheter, is used together with a restorator in case of balloon kyphoplasty. Instruments that are used for providing a path to a bone each comprises a needle generally composed of a pipe and a steel wire, a wire pin, a cannula, and an expander. Further, they may additionally comprise a spacer, a filler, a pusher, etc.

The balloon kyphoplasty is performed by, for example, inserting a long thin special tube into a compression fractured region, inserting a balloon through the tube and expanding it to a normal height, and then removing the balloon and filling the thus-formed space with a bone-filling material (e.g. bone cement and bone substitute).

The procedures of the conventional kyphoplasty can be known from the descriptions of U.S. Pat. No. 6,241,734, which is incorporated herein by reference. In detail, the balloon kyphoplasty is performed by: inserting a needle with a pointed tip (in an operation) into the vertebral body through surgical cut from the outside of the body and removing the steel wire out of the pipe of the needle; thereafter, inserting a wire pin into the pipe and pulling the pipe out of the body; thereafter, inserting a cannula and an expander into the body with the guidance by the wire pin and expanding the inside of the vertebral body by operating the expander from the outside, and then separating the expander from the body; thereafter, inserting a spacer into the cannula and ensuring an inner space using a drill-shaped tip such that cavity-forming instruments, such as a balloon catheter, can be smoothly inserted; thereafter, equipping a restorator with a balloon catheter and inserting the tip of the catheter with a balloon into the cannula, and then expanding the balloon inside the vertebral body using pressure from the restorator; thereafter, repeatedly inserting each of a plurality of fillers (disposal) into the cannula and injecting the bone-filling material into the expanded space inside the vertebral body by pushing the inside of the pipe-shaped filler using a pusher, whereby the operation is completed.

However, the catheter accessories that are used in the surgical operation in the related art have some problems and inconvenience.

For example, the needle that is inserted into the surgical patient's body through the skin by an operator is commonly formed of a probe with a pointed tip and inserted through the vertebral body by a surgical hammer or an electric tool. However, after being inserted to the vertebral body, the needle is difficult to be separated from the bone because the compounds of inorganic substances and organic substances act like a concrete. In order to overcome this problem, atypical methods such as using a specific separating device or hitting the needle in the opposite direction to the hitting direction to be inserted are used, which is inconvenient for the operator and reduces operational accuracy.

Further, in the process of inserting the cannula and the expander into the body using the wire pin as a guide and then expanding the inside of the vertebral body by operating the expander from the outside, it is not easy to stably fix the expander to and/or to separate the expander from and the cannula, which causes inconvenience and limits.

In addition, the spacer used for ensuring a space inside the vertebral body has a drill-shaped tip, but the tip causes unnecessary cutting while cutting into the tissue of the bone and reduces efficiency of the operation.

The above information disclosed in this Background section is only for enhancement of understanding of the background of the invention and therefore it may contain information that does not form the prior art that is already known in this country to a person of ordinary skill in the art.

### SUMMARY

Therefore, in order to overcome the above problems, an object of the invention is to provide an apparatus for treating a bone that makes it possible to easily combine/separate an expander with/from a cannula. Another object of the invention is to provide an apparatus for treating a bone that makes it possible to easily separate a needle or a steel wire in the needle from the bone. Another object of the invention is to provide an apparatus for treating a bone that can prevent unnecessary cutting and can easily ensure an insertion space for a cavity-forming instrument inside the bone. An additional object of the invention is to provide an apparatus for treating a bone that can improve convenience in consideration of an ergonomical outer structure.

In order to achieve the above objects, the prevent invention provides a new apparatus including an expander and a cannula with an improved coupling structure. Further, the invention provides an apparatus that additionally comprises a needle with an improved coupling structure with other components. Further, the invention provides an apparatus that additionally comprises a spacer with an improved threaded portion. Further, the invention provides an apparatus that additionally comprises other instruments, such as a guide, a filler, and a pusher. In addition, the invention provides an apparatus for treating a bone comprising a needle assembly that can easily be separated from the bone, and an apparatus for treating a bone comprising a spacer assembly with an improved structure such that it does not incur unnecessary cutting.

According to the apparatuses for treating a bone, e.g., percutaneous balloon vertebroplasty, it is possible to provide an approach path through a bone in a more effective and simpler manner and ensure a space inside a bone before using an instrument for forming a space using a balloon to insert a bone-filling material. That is, it is possible to stably fix the expander to the cannula, easily separate the expander from the bone or the cannula, and simplify the operational procedure by providing a dockable expander and cannula. Further, it is possible to easily separate the needle from the bone. Further, unnecessary cutting is not incurred when operating the spacer, such that efficiency of work can be improved while easily ensuring the space for smoothly inserting a cavity-forming instrument into the bone. As a result, the operating time is reduced and the operational completeness can be improved.

### BRIEF DESCRIPTION OF THE DRAWINGS

The above and other features and advantages of the present invention will become more apparent by describing in detail preferred embodiments thereof with reference to the attached drawings in which:

FIG. 1 is a perspective view of a cannula and an expander assembly in a docked/combined state according to an embodiment of the invention;

FIG. 2 is an exploded view of a cannula, an expander assembly, and a guiding instrument according to an embodiment of the invention;

FIG. 3 is a perspective view of a needle assembly in a combined state according to an embodiment of the invention;

FIG. 4 is a cross-sectional view of a needle assembly according to an embodiment of the invention and FIG. 5 is an enlarged view of the tip of a steel wire according to a one embodiment of the present invention;

FIG. 6 is an exploded view of a needle assembly according to an embodiment of the invention;

FIG. 67 is a perspective bottom view of a coupling portion of a steel wire assembly and an expander assembly;

FIG. 8 is a schematic view illustrating a coupling portion in a combined state;

FIG. 9 is a schematic view illustrating a coupling portion in a separated state;

FIG. 10 is an enlarged view of a spacer assembly according to an embodiment of the invention;

FIG. 11 is a cross-sectional view of a filler and a pusher according to an embodiment of the invention, before they operate;

FIG. 12 is a cross-sectional view of the filler and pusher of FIG. 11 when they operate; and

FIG. 13 is a perspective view of a needle assembly, a guiding instrument, a cannula, an expander assembly, a spacer assembly, a filler assembly and a pusher assembly according to an embodiment of the invention.

### DETAILED DESCRIPTION

Hereinafter reference will now be made in detail to various embodiments of the present invention, examples of which are illustrated in the accompanying drawings and described below. While the invention will be described in conjunction with exemplary embodiments, it will be understood that present description is not intended to limit the invention to those exemplary embodiments. On the contrary, the invention is intended to cover not only the exemplary embodiments, but also various alternatives, modifications, equivalents and other embodiments, which may be included within the spirit and scope of the invention as defined by the appended claims.

As discussed above, the present invention provides an apparatus for treating a bone. According to an embodiment, the apparatus may comprise a cannula 300 and an expander assembly 400. The cannula 300 suitably comprises a pipe 330 and a handle 311 connected to an end of the pipe 330 and having a receiving groove 312. The expander 400 suitably comprises an expanding pipe 430 which can be inserted into the pipe 330 of the cannula 300 and a head 421 that is connected to an end of the expanding pipe 430 and is configured to be received in the receiving groove 312 through a coupling means.

The cannula 300 can be suitably docked with the expander assembly 400, such that the operation is simplified by a one-step method and thereby the operation time is reduced.

Suitably, the coupling means may comprise a guiding protrusion 313 that protrudes from a portion of the inner side of the receiving groove 312 and a seat 429 that is formed at a portion of the bottom of the head 421 to lock the guiding protrusion 313.

Preferably, the coupling means may further comprise a locking projection 428 protruding from a portion of the bottom of the head 421 to prevent the guiding protrusion 313 from being unlocked from the seat 429.

Suitably, the coupling means may further comprise a guiding means for facilitating the guiding protrusion 313 to be locked into the seat 429. The guiding means may be any means known in the art. An example of the guiding means comprises a fixing groove 315 formed at a side portion of the receiving groove 312 and a fixing protrusion 423 protruding from a side portion of the head 421 so as to fit into the fixing groove 315.

The guiding means may further comprise a guide slope 427 that is formed at a predetermined angle on a portion of the bottom of the head 421 to facilitate the guiding protrusion 313 to be slid into the seat 429.

According to the apparatuses, the cannula is docked with the expander by the coupling means, such that the expander can be easily and stably fixed to the cannula during the operation and also easily separated from the cannula or a bone.

In an embodiment, the handle 311 of the cannula 300 can receive the head 421 of the expander at the receiving groove 312 which is formed at a upper portion. Further, the handle 311 having the receiving groove 312 has an insertion hole 3 16 at the center portion thereof, so that the expanding pipe 430 can be put into the pipe 330. A curved portion 314 may be formed on a portion of the bottom of the handle 311 to provide stability in the operation and a fixing member 318 may be provided to fix the pipe 330 to the handle 311. The materials by which the fixing member 318 and the handle 311 are made of or from are not limited. Suitably, the handle 311 can be made of materials known in the art, for example, acrylonitrile butadiene styrene copolymer, polypropylene resin, nylon, acetal resin, polyethylene resin, polyethylene terephthalate resin, high impact polystyrene (HIPS) resin, acrylonitrile styrene copolymer, heat resistant resin etc.

The pipe 330 of the cannula is hollow to insert the expanding pipe 430 and has a larger diameter than the expanding pipe 430. For example, the diameter of the pipe 330 may be in the range of 2.5 to 7.5 mm. The pipe 330 can be made of any material known in the art, for example, a stainless steel, aluminum, titanium etc, but not limited thereto. A marking 331, which indicates the insertion depth of the cannula, is provided on a side of the pipe 330.

According to an embodiment of the invention, the head 421 of the expander 400 may be provided with a grip 424 having a plurality of grooves at the front and rear sides to allow the head 421 to be easily turned while preventing a hand gripping of the instrument from slipping. An outer groove 425 may be formed below the grip 424 to provide a stable grip and reduce the entire weight. The head 420 may be made of, for example, acrylonitrile butadiene styrene copolymer, polypropylene resin, nylon, acetal resin, polyethylene resin, polyethylene terephthalate resin, high impact polystyrene (HIPS) resin, acrylonitrile styrene copolymer, and etc, but not limited thereto.

Further, the expanding pipe 430 of the expander 400 is hollow to insert another instrument. The expanding pipe 430 has a diameter smaller than the pipe 330 of the cannula and larger than another instrument (e.g. guiding instrument) that can be inserted therein. It is preferable that the frontal end (in an operation) of the expanding pipe 430 is tapered to be easily inserted into the bone. The expanding pipe 430 can be made of a material, for example a stainless steel, aluminum, titanium etc, but not limited thereto.

According to an embodiment of the invention, the head 421 has a hole 426 at the center portion thereof, so that it is connected to the expanding pipe 430 to allow another instrument used as a guide to the bone to be passed through the expanding pipe 430 and the head 421. The instrument used as a guide to the bone may be an instrument known in the art, for example, a guiding instrument 200 formed of a wire pin.

In an embodiment, the apparatus may further comprise a needle assembly 100 that is to establish the initial path from the skin to the bone.

Preferably, the needle assembly 100 may comprise a pipe assembly 110 and a steel wire assembly 120. The pipe assembly 110 comprises a pipe 130 and a handle 111 connected to an end of the pipe 130 and having a receiving groove 112. The steel wire assembly 120 includes a steel wire 122 that is able to be inserted into the pipe 130 of the pipe assembly 110 and a head 121 that is connected to an end of the steel wire 122 and is configured to be received in the receiving groove 112 through a coupling means.

Suitably, the coupling means may further comprise a guiding protrusion 113 protruding from a portion of the inner side of the receiving groove 112 and a seat 129 formed on a portion of the bottom of the head 121 to lock the guiding protrusion 113.

Preferably, the coupling means may further comprise a locking projection 128 protruding from a portion of the bottom of the head 121 to prevent the guiding protrusion 113 from being unlocked from the seat 129.

Suitably, the coupling means may further comprise a guiding means that facilitates the guiding protrusion 113 to be locked into the seat 129.

The guiding means may be any means known in the art. According to an embodiment, the guiding means comprises a fixing groove 115 formed at the sides of the receiving groove 112 and a fixing protrusion 123 protruding from the sides of the head 121 and fitted into the fixing groove 115.

Further, the guiding means may further comprises a guide slope 127 that is formed at a predetermined angle on a portion of the bottom of the head 121 to facilitate the guiding protrusion 113 to be slid to the seat 129.

As described above, the needle assembly may suitably comprise the pipe combined with the steel wire by the coupling means and the receiving groove, such that the needle or the steel wire in the needle can be easily separated from the bone and the operating time can be reduced.

According to an embodiment, the handle 111 of the pipe assembly 110 can receive the head 121 of the steel wire assembly 120 at the receiving groove 112 formed at the upper portion. Further, the handle 111 having the receiving groove 112 has an insertion hole 116 at the center portion thereof, so that the steel wire 122 of the steel wire assembly can be inserted into the pipe 130. A curved portion 114 may be formed on the bottom of the handle 111 to provide stability in the operation and a fixing member 118 may be provided to fix the pipe 130 to the handle 111. The fixing member 118 may be any one as long as it can perform the fixing function. Further, the handle 111 can be made of desired materials known in the art, for example, acrylonitrile butadiene styrene copolymer, polypropylene resin, nylon, acetal resin, polyethylene resin, polyethylene terephthalate resin, high impact polystyrene (HIPS) resin, acrylonitrile styrene copolymer, and etc, but not limited thereto.

The pipe 130 of the pipe assembly 110 is hollow to insert the steel wire 122 of the steel wire assembly 130 and has a larger diameter than the steel wire 122. For example, the diameter of the pipe 130 is in the range of 1 to 6 mm. The pipe 130 can be made of a material known in the art, for example, stainless steel, aluminum, titanium etc, but not limited thereto. It is preferable that the frontal end (in an operation) of the pipe 130 is tapered (tapered portion 117) to be easily inserted into the bone.

The head 121 of the steel wire assembly 120 is provided with a grip 124 having a plurality of grooves at the front and the rear sides to allow the head 121 to be easily turned while preventing a hand gripping the instrument from slipping. An outer groove 125 may be formed below the grip 124 to provide a stable grip and reduce the entire weight. For example, the head may be made of acrylonitrile butadiene styrene copolymer, polypropylene resin, nylon, acetal resin, polyethylene resin, polyethylene terephthalate resin, high impact polystyrene (HIPS) resin, acrylonitrile styrene copolymer, and etc, but not limited thereto.

It is preferable that the frontal end (in an operation) of the steel wire 122 of the steel wire assembly 120 is sharp and pointed to be easily inserted into the bone. For example, the other end of the steel wire 122 can be in the shape of a pyramid such as triangular, quadrangular, pentagonal, or hexagonal pyramid and cheek-cut cylinder, as illustrated in FIG. 5. The steel wire 122 can be made of a material, for example, a stainless steel wire, and a titanium stick, and etc, but not limited thereto.

Preferably, the apparatus may further comprise an instrument used for a guide into the bone. An example of such instrument is the guiding instrument 200 formed of a wire pin.

According to an embodiment, when the apparatus comprises the cannula 300, the expander assembly 400, and the needle assembly 100, the instrument used for a guide into the bone is inserted into the pipe 130 of the pipe assembly 110 after the steel wire assembly 120 is removed from the needle assembly 100. After the pipe assembly 110 is removed, the cannula 300 docked with the expander 400 guided by the guide instrument 200 is inserted into the bone. Accordingly, it is preferable that the guiding instrument 200 has a diameter smaller than the pipe 130 of the pipe assembly 110 or the expanding pipe 430.

Further, the apparatus may further comprise an instrument for forming an insertion space for the cavity-forming instrument inside the vertebral body.

According to an embodiment of the invention, a spacer instrument 500 is used as the instrument for forming the insertion space for the cavity-forming instrument. The spacer assembly 500, for example, comprises a steel wire 522 having a treaded portion 520 at an end thereof and being able to be inserted in the pipe 330 of the cannula 300 and a handle 511 connected to the other end of the steel wire 522. The spacer assembly 500 is inserted into the pipe 330 of the cannula 300 after the expander assembly 400 is undocked from the cannula 300.

Meanwhile, the cavity-forming instrument used in the invention may be any one known in the art, and for example, may be a balloon catheter having a structure known in the art.

According to an embodiment of the invention, the threaded portion 520 is formed of a double- to fourfold-thread having a phase difference of 90° to 180° such that a distance that the thread moves forward in one turn thereof is equal to two to four times the axial distance between any point on a thread and a corresponding point on the next thread. Preferably, the threaded portion 520 may be formed of a threefold-thread having the phase difference of 120° such that a distance that the thread moves forward in one turn thereof is equal to three times the axial distance between any point on a thread and a corresponding point on the next thread.

With the above-described threaded portion 520, it is possible to easily ensure the insertion space for the cavity-forming instrument, such as the balloon catheter, inside the bone without causing excessive cutting that has been a problem in the drill-bit type in the related art.

According to an embodiment, left and right symmetric protrusions are provided at the upper portion of the handle 511 of the spacer assembly 500 and a curved grip 524 is formed at the lower portion to allow an operator to easily turn the handle 511.

Further, the apparatus may further comprise a filler assembly 600 and a pusher assembly 700. The filler assembly 600 is configured to accommodate a bone-filling material to be inserted therein. The pusher assembly 700 functions to push the bone-filing material inserted in the filler assembly 600 toward the cavity formed within a bone.

The filler assembly 600 and the pusher assembly 700 may be ones known in the art. Medical bone cement or known bone substitute material can be used as the bone-filling material.

According to an embodiment of the invention, the filler assembly 600 comprises a pipe 630 that is configured to be inserted into the pipe 330 of the cannula 300 and a handle 611, which has a cushioning member 626 with a predetermined-height stepped portion such that the pusher assembly 700 can be inserted, at an end thereof, and the pipe 630 at the other end.

The filler assembly 600 can be inserted into the cannula 300 after the spacer assembly 500 is separated or undocked from the cannula 300. The handle 611 of the filler assembly 600 may have left and right protrusions that are symmetrical and a curved grip 624 at the bottom. The cushioning member 626 can be made of any material including acrylonitrile butadiene styrene copolymer, polypropylene resin, nylon, acetal resin, polyethylene resin, polyethylene terephthalate resin, high impact polystyrene (HIPS) resin, acrylonitrile styrene copolymer, and etc, but not limited thereto. The height of the step of the cushioning member 626 can be freely selected by those skilled in the art, and for example, in a range of 2 to 15 mm.

According to an embodiment of the invention, the pusher assembly 700 comprises a pin 722 that is configured to be inserted in the filler assembly 600 and a handle 711 that is connected to one end of the pin 722.

The handle 711 is not limited in the shape, as long as it is suitable for a cautious operation, and for example, may be formed in a snow man shape.

According to an embodiment of the invention, an apparatus of the invention may comprise: a needle assembly 100 comprising a pipe assembly 110 having a pipe 130 and a handle 111 connected to an end of the pipe 130 and having a receiving groove 112, and a steel wire assembly 120 that has a steel wire 122 configured to be inserted in the pipe 130 of the pipe assembly 110 and a head 121 disposed at an end of the steel wire 122 and configured to be received in the receiving groove 112 by a coupling means; a guiding instrument 200 configured to be inserted in the needle assembly 100, for a guide into the bone; a cannula 300 being able to be inserted into the bone while being guided by the guiding instrument 200 after the needle assembly 100 is separated from the guiding instrument 200, and having a handle 311 connected to an end of the pipe 330 and having a receiving groove 312; a expander 400 comprising an expanding pipe 430 that is able to be inserted in the pipe 330 of the cannula 300 and a head 421 disposed at an end of the expanding pipe 430 and is configured to be received in the receiving groove 312 by the coupling means; a spacer assembly 500 being able to be inserted into the cannula 300 after the expander 400 is separated or undocked from the cannula 300 and having a steel wire 522 having a threaded portion 520 at an end thereof and a handle 511 at the other end; a filler assembly 600 being able to be inserted into the cannula 300 after the spacer assembly 500 is separated and having a cushioning member 626 at an end thereof and a handle 611 at the other end where a pipe 630 is connected; and a pusher assembly 700 having a pin 722 that is configured to be inserted in the filler assembly 600 filled with a bone-filling material and a handle 711 disposed at an end of the pin 722.

Further, the apparatus may suitably comprise an improved needle assembly 100 that is easily separated from the bone.

In detail, the apparatus comprises a needle assembly 100 including a pipe assembly 110 having a pipe 130 and a handle 111 connected an end of the pipe 130 and having a receiving groove 112, and a steel wire assembly 120 having a wire 122 configured to be inserted in the pipe 130 of the pipe assembly 110 and a head 121 disposed at an end of the steel wire 122 and configured to be received in the receiving groove 112 by a coupling means, in which the structures of the pipe assembly 110 and the steel wire assembly 120 of the needle assembly 110 are the same as those as described above.

Suitably, the apparatus may further comprise a cannula and an expander assembly known in the art, and may further comprise one or more of a guiding instrument, a spacer assembly, a filler, and a pusher assembly that are known in the art. The cannula and the expander assembly may have the same structures as in the invention.

Further, an apparatus may further comprise a spacer assembly 500 for ensuring an insertion space for a cavity-forming instrument inside the bone.

Suitably, the spacer assembly 500 comprises a steel wire 522 having a threaded portion 520 at an end and a handle 511 connected to the other end of the steel wire 522. Further, the threaded portion 520 may be formed of a double- to fourfold-thread having a phase difference of 90° to 180° such that a distance that the thread moves forward in one turn thereof is equal to two to four times the axial distance between two points corresponding to the adjacent threads (the lead is 2˜4 times larger than the pitch).

The apparatus for treating a bone including the spacer assembly 500 may further comprise one or more of a needle assembly, a cannula assembly, an expander assembly, a guiding instrument, a filler assembly, a pusher assembly that are known in the art. The needle, cannula, and expander may have the same structures as in the invention.

Hereinafter, the apparatuses will be described in more detail with regard to the accompanying drawings.

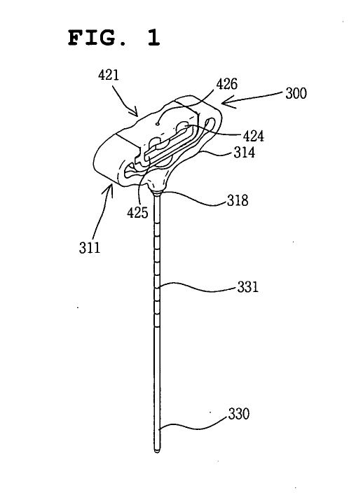

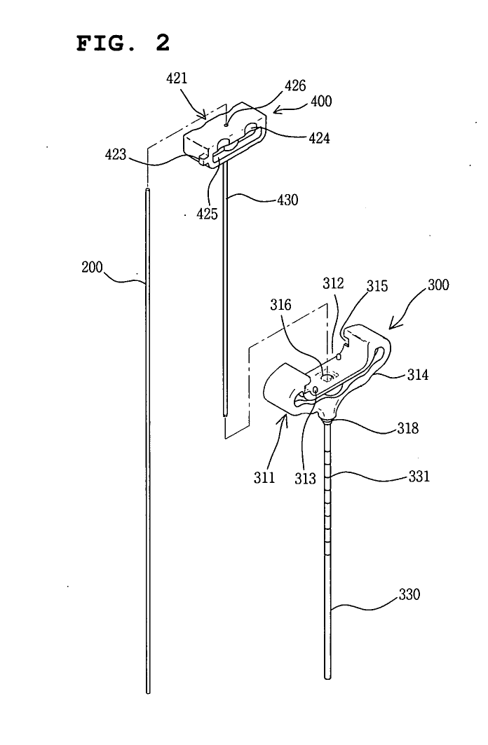

FIGS. 1 and 2 are views showing the structures of a cannula and an expander assembly according to an embodiment of the invention.

The cannula 300 includes a handle 311 having a receiving groove 312 on a upper portion thereof to receive a head 421 of an expander assembly 400, and a curved portion 314 at a bottom portion thereof. Fixing grooves 315 are formed at both sides of the receiving groove 312. Fixing protrusion 423 are formed at the head 421 of the expander 400, which are fitted into the fixing grooves 315. Further, in a plan view, a guiding protrusion 313 is formed on the inner side of the receiving groove 312 to easily dock/undock the cannula 300 with/from the expander 400 and an insertion hole 316 is formed at the center portion of the handle 311 having the receiving groove 312 to insert an expanding pipe 430 of the expander assembly into a pipe 330. The pipe 330 is fixed to the bottom of the handle 311 by a fixing member 318 and a marking 331 that indicates the insertion depth is provided on a side of the pipe 300.

Further, the expander 400 includes the head 421 and the expanding pipe 430. Similar to the curved portion 314 formed to provide stability when operating the cannula 300, the head 421 has a grip 424 having a plurality of grooves at the front and the rear sides to allow a hand gripping the instrument to easily turn the instrument without slipping. An outer groove 425 is formed below the grip 424 to provide a stable grip and reduce the weight of the head. The fixing protrusions 423 are formed at the left and right sides of the head 421 and fitted into the fixing grooves 315 of the cannula 300. Further, the head 421 has a hole 426 at the center portion thereof to be connected with the expanding pipe 430 such that a guiding instrument 200 can pass through the expanding pipe 430 and the head 421. The expanding pipe 430 is hollow to allow the guiding instrument 200 to slip into the inside and is tapered at the frontal end.

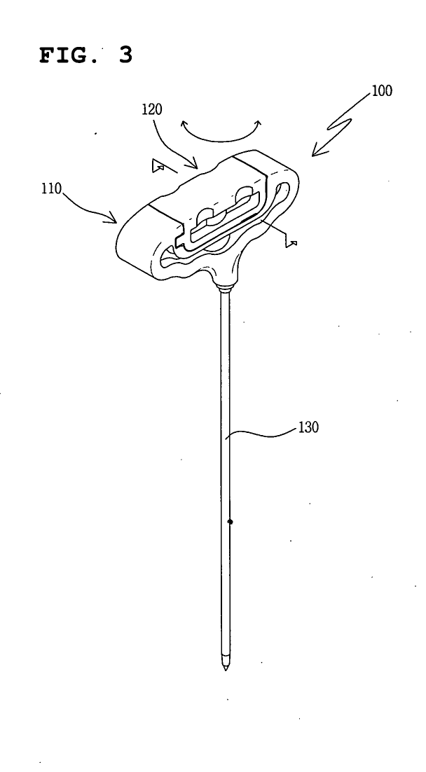

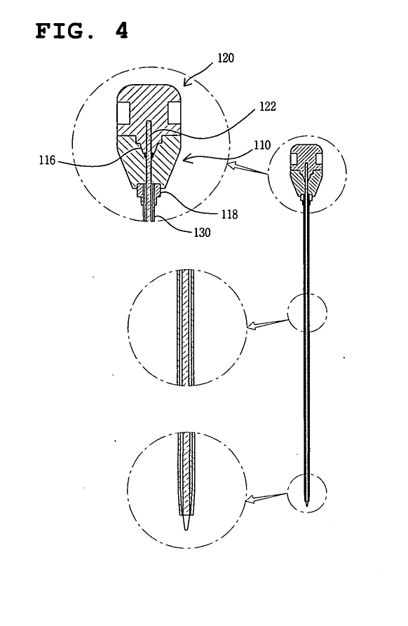

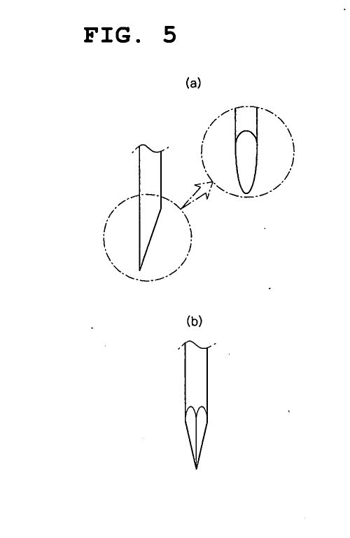

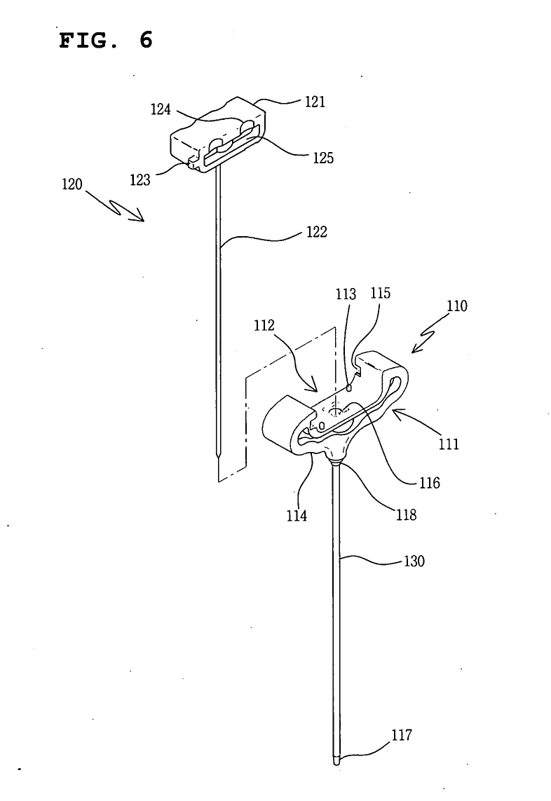

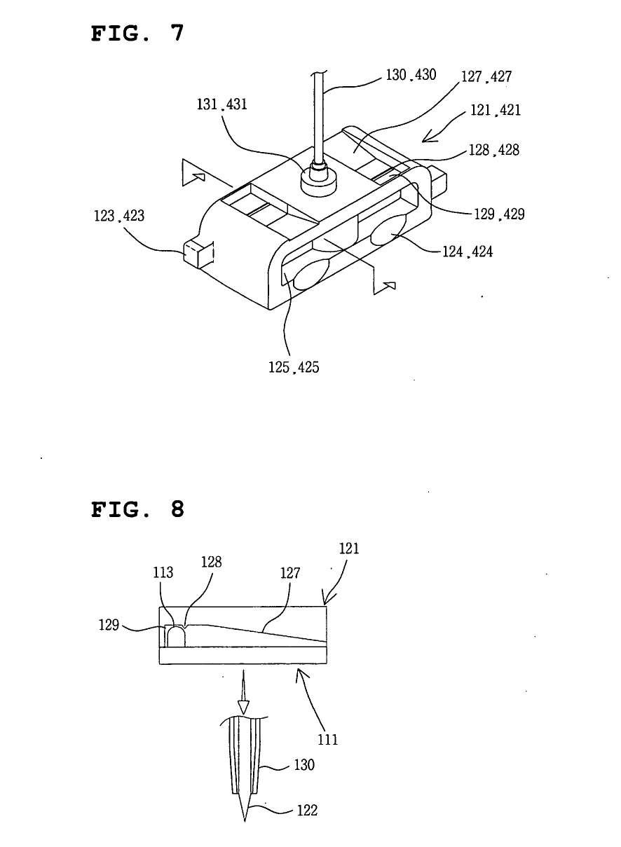

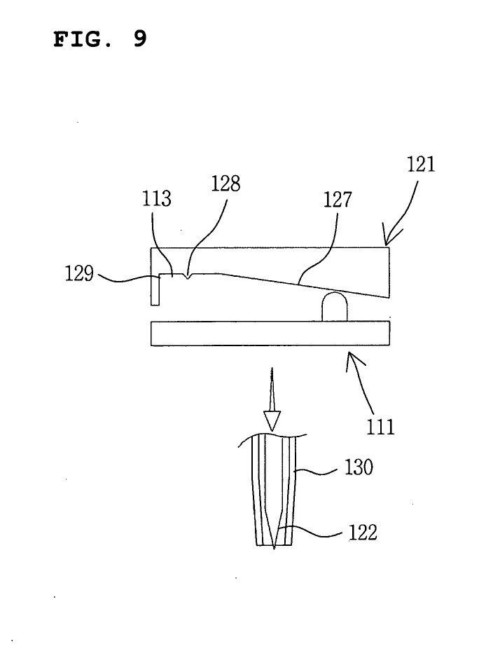

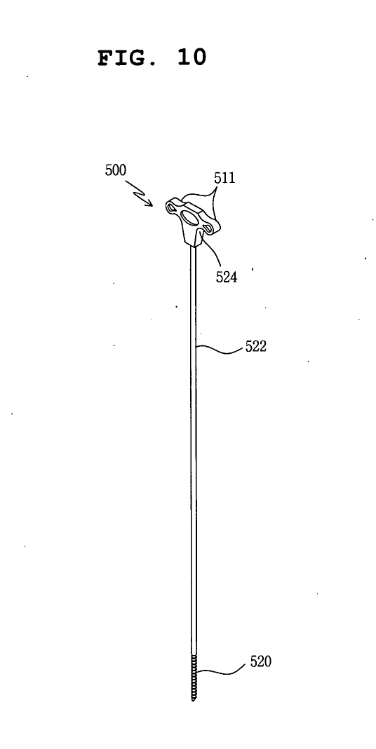

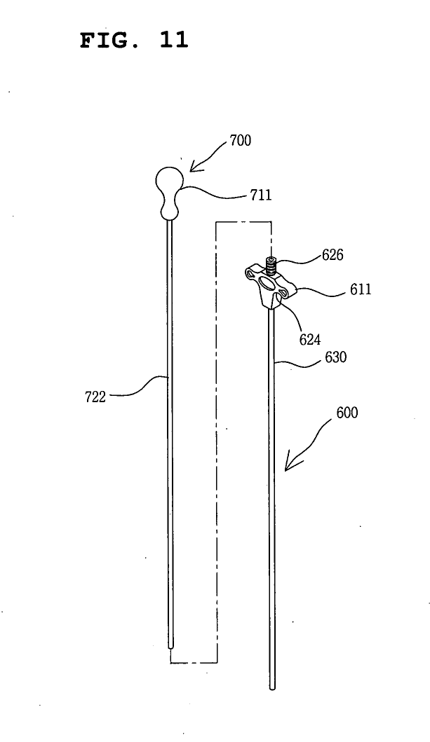

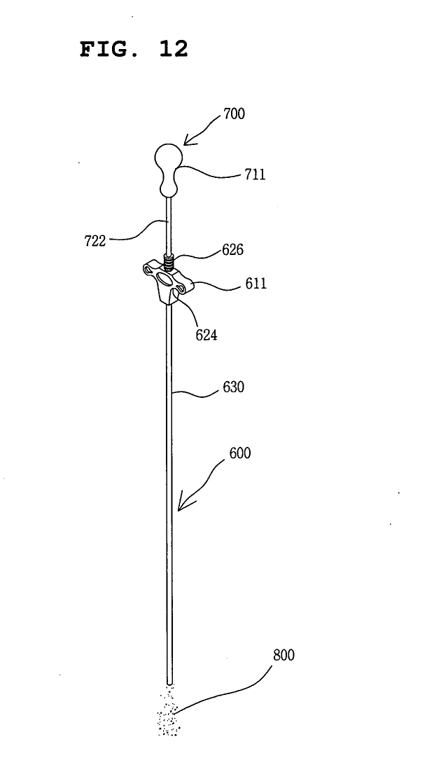

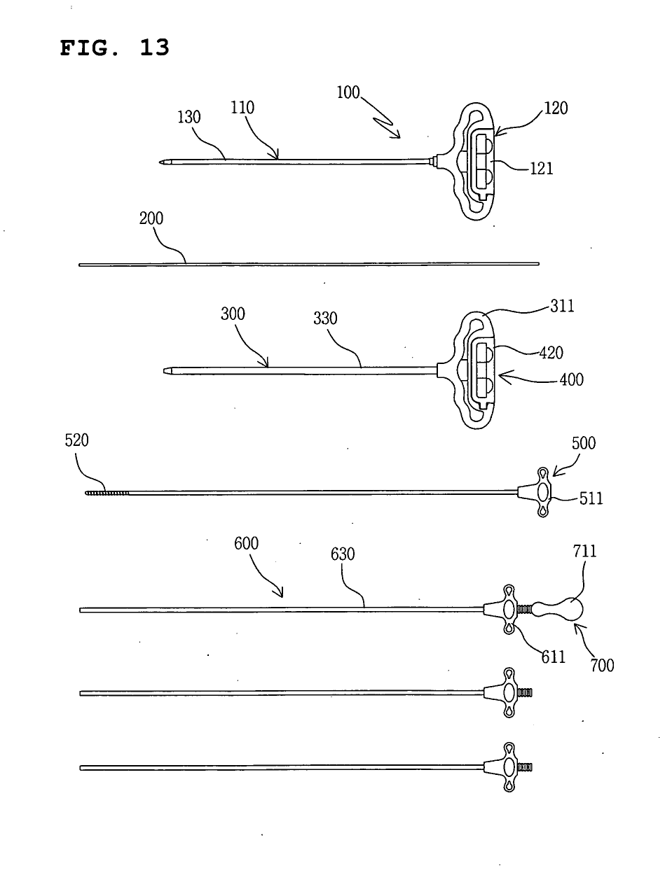

FIGS. 3 to 6 are views showing the structures of a needle assembly 100 according to an embodiment of the invention.

Referring to FIGS. 3 to 6, the needle assembly 100 comprises: a pipe assembly 110 comprising a pipe 130 and a handle 111 connected to an end of the pipe 130 and having a receiving groove 112; and a steel wire assembly 120 comprising a steel wire 122 that is configured to be inserted into the pipe 130 of the pipe assembly 110 and a head 121 that is connected to an end of the steel wire 122 and is configured to be received in the receiving groove 112 through a coupling means.

The handle 111 of the pipe assembly 110 has a receiving groove 112 at a upper portion thereof to receive the head 121 of the steel wire assembly 120, and a curved portion 114 at a bottom portion. Fixing grooves 115 are formed at both sides of the receiving groove 112. Fixing protrusions 123 are formed at the head 121 of the steel wire assembly 120, which are fitted into the fixing grooves 115. Further, in a plan view, guiding protrusions 113 are formed on the inner side of the receiving groove 112 to easily dock/undock the pipe assembly 110 with/from steel wire assembly 120 and an insertion hole 116 is formed at the center portion of the handle 111 having the receiving groove 112 to insert an a wire 122 of the steel wire assembly into a pipe 130. The pipe 130 is fixed to the bottom of the handle 111 by a fixing member 118 and the pipe 130 has a tapered portion 117 at the frontal end (in an operation).

The steel wire assembly 120 includes the head 121 and the steel wire 122. Similar to the curved portion 114 formed to provide stability when operating the pipe assembly, the head 121 has a grip 124 having a plurality of grooves at the front and rear sides to allow a hand gripping the instrument to easily turn the instrument without slipping. An outer groove 125 is formed at the lower portion of the grip 124 to provide a stable grip and reduce the weight of the head. The fixing protrusions 123 are formed at the left and right sides of the head 121 and fitted into the fixing grooves 115 of the pipe assembly 110. The front end of the steel wire 122 is sharp and pointed in the shape of a cheek-cut cylinder or a pyramid, to be easily inserted into the vertebra.

FIG. 7 is a perspective bottom view illustrating the configuration of a coupling portion that is a portion of the steel wire assembly 120 or the expander assembly 400, and FIGS. 8 and 9 are views showing that the coupling portion of a needle assembly where the pipe assembly and the steel wire assembly are combined/docked together (FIG. 8) and separated/undocked from each other (FIG. 9).

The steel wire assembly 120 and the expander assembly 400, as shown in FIG. 7, each have, at their bottoms: guide slopes 127, 427 that guide the guiding protrusions 113, 313 of the pipe assemblies 110 to slide toward seats 129, 429 to be docked/combined with the pipe assembly 110 or the cannula 300; locking projections 128, 428 that prevent the guiding protrusions 113, 313, that lead the docking through a moving along the guide slopes 127, 427, from unlocking by a moving backward; and the seats 129, 429 where the guiding protrusions 113, 313 passing the locking projections 128, 428 are seated. With this structure, it is possible to easily separate the steel wire from the needle or the bone, and stably combine or dock the expander with the cannula and easily separate them.

An exemplary operation of the guiding protrusions 113, 313, the guide slopes 127, 427, and the locking projections 128, 428 are illustrated in FIGS. 8 and 9, with reference to the needle assembly 100.

As shown in FIG. 8, when the guiding protrusion 113 of the pipe assembly 110 is guided along the guide slopes 127 of the steel wire assembly 120 and pass the locking projections 128, it is placed on the seat 129 of the steel wire assembly 120 and the docking of the pipe assembly 110 and the steel wire assembly 120 is completed and the steel wire 122 of the steel wire assembly 120 becomes to protrude out of the pipe 130 of the pipe assembly 110 as illustrated in the lower part of FIG. 8. With this structure, the steel wire assembly 120 can perform the initial establishment of a path to the vertebral body.

In order to pull the steel wire 122 run down to the bone out of the vertebral body, the operation illustrated in FIG. 9 is performed. That is, as an operator takes hold of and turns the grip 124 of the steel wire assembly 120 docked with the pipe assembly 110, the guiding protrusion 113 is moved along the guide slopes 127 and the steel wire assembly 120 is separated/undocked from the pipe assembly 110. Further, the steel wire 122 of the steel wire assembly 120 is pulled out of the vertebral body by moving it upward out of the pipe 130 of the pipe assembly 110.

FIG. 10 is an enlarged view showing the structure of a spacer assembly 500 according to an embodiment of the invention. The space assembly 500, as described above, is provided to form a space to smoothly insert a cavity-forming instrument, such as a balloon catheter, into the vertebra.

The space instrument 500 includes a handle 511 and a steel wire 522. The handle 511 has protrusions, which are bilaterally symmetric, at the upper portion and a curved grip 524 at the bottom thereof. According to this structure, an operator can easily twist the grip of the hand taking hold of the handle 511 with fingers in the grip 524. The steel wire 522 is connected to the lower end of the handle 511 with the grip 524. A threaded portion 520 is formed at the front end (in an operation) of the steel wire 522 to form a space inside the bone. In order not to cause excessive cutting that may occur when using the drill-bit type in the related art, the threaded portion 520 is formed of a triple-thread such that it has a phase difference of 120° and the axial distance that the thread moves in one turn around the screw is equal to three times the axial distance between two points corresponding to the adjacent threads.

FIGS. 11 and 12 are cross-sectional views illustrating a filler assembly 600 and a pusher assembly 700 of an apparatus for treating a bone according to an embodiment of the invention before and after they operate.

The filler assembly 600 includes a handle 611, a pipe 630, and a cushioning member 626. The handle 611, similar to the handle 511 of the spacer assembly 500, has protrusions, which are bilaterally symmetric at the upper portion and a curved grip 624 at the bottom thereof. The cushioning member 626, which has a predetermined-height stepped portion such that a pin 722 of the pusher assembly 700 can be inserted, is formed on the upper end of the handle 611 with the grip 624. The pipe 630 that guides the pin 722 and is inserted into the pipe 330 of the cannula is connected to the lower end of the handle 611.

Further, the pusher assembly 700 has a handle 711 at the upper portion and the pin 722 connected to the lower end of the handle 711. The handle 711 has a snowman shape.

According to an exemplary embodiment, after inserting a bone-filling material 800, e.g. medical cement, into the inside of the pipe 630 of the filler assembly 600 and pushing the material with the pin 722 of the pusher assembly 700, cement 800 inserted within the pipe 630 is displaced out the frontal end of the pipe 630 and into the cavity, whereby it is possible to fill the cement into the cavity formed in the vertebral body.

As shown in FIG. 13, an apparatus according to an embodiment may comprise: a needle assembly 100, a guiding instrument 200, a cannula 300, an expander assembly 400, a spacer assembly 500, a filler assembly 600, and a pusher assembly 700.

The needle assembly 100 comprises: a pipe assembly 110 formed of stainless steel or aluminum and having a pipe 130 and a handle 111 with a receiving groove 112 and connected to an end of the pipe 130; and a steel wire assembly 120 including a steel wire 122 inserted into the pipe 130 of the pipe assembly 110 and a head 121 connected with an end of the steel wire 122 and is configured to be received in the receiving groove 112 through a coupling means.

The guiding instrument 200 is formed of a wire pin and configured to be inserted into the needle assembly 100.

The cannula 300 comprises a pipe 330 and a handle 311 having a receiving groove 312 and connected to an end of the pipe 330, and is guided by the guiding instrument 200 to be inserted into the bone after the needle assembly 100 is separated from the guiding instrument 200.

The expander 400 has an expanding pipe 430 being able to be inserted in the pipe 330 of the cannula 300 and a head 421 connected to an end of the expanding pipe 430 and is configured to be received in the receiving groove 312 by a coupling means to be in a docked state.

The spacer assembly 500 has a steel wire 522 having a threaded portion 520 at an end thereof and a handle 511 at the other end, and is inserted into the cannula 300 after the expanding assembly 400 is separated/undocked from the cannula 300.

A plurality of the filler assemblies 600 each have a cushioning member 626 at an end thereof and a handle 611 connected to a pipe 630 at the other end, and are inserted into the cannula 300 after the spacer assembly 500 is separated.

A plurality of the pusher assemblies 700 each have a pin 722 inserted into the filler assemblies 600 filled with a bone-filling material and a handle 711 connected to an end of the pin 722.

A general operation of vertebroplasty using the apparatus is exemplified hereafter.

In the first step, the surgical patient's back is cut open and the steel wire assembly of the needle assembly is inserted into the vertebra through the subcutaneous tissue, in which a needle including a pipe is also inserted into the vertebra.

In the second step, the inserted steel wire assembly of the needle is separated from the pipe, a wire pin is inserted into the pipe and positioned in place, and the inserted pipe is removed from the vertebra such that the entire needle is separated.

In the third step, the cannula is inserted while being guided by the wire pin partially inserted in the vertebra, in which an expander with the tapered front end is inserted in the cannula, which is inserted, for easy insertion.

In the fourth step, the expander and the wire pin are separated or undocked from the cannula and the spacer is inserted into the cannula to form a space such that a balloon catheter can be smoothly inserted.

In the fifth step, the catheter with a balloon at the front end (in an operation) is inserted into the vertebra through the cannula, a separate cylinder is attached to the outer side of the catheter, and then the balloon of the catheter is expanded by operating the attached cylinder.

In the sixth step, after the body of the vertebra returns to the initial shape, the balloon catheter is separated from the cannula, a bone-filling material (e.g. medical bone cement) is filled into the pipe of the filler and inserted into the cannula; thereafter, the pusher is pushed into the pipe of the filler from the outside such that the cement if injected into the space formed by the balloon inside the vertebral body.

As cement is injected into the fractured vertebra by the above steps and then hardened therein, the body of the vertebra can return to the initial shape.

The present invention provides an apparatus for treating a bone that provides a path from the skin to the bone, which gives satisfaction to an operator and a surgical patient after the operation and makes it possible to perform efficient vertebroplasty using a balloon catheter by removing problems due to inconvenience in the operation and providing an improved functional structure.

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

What is claimed is:

1. An apparatus for treating a bone that provides a path from a skin to a bone, comprising:
a cannula (300) which comprises a pipe (330) and a handle (311) connected to an end of the pipe (330) and having a receiving groove (312); and
an expander (400) which comprises an expanding pipe (430) being able to be inserted into the pipe (330) of the cannula (300) and a head (421) connected to an end of the expanding pipe (430) and configured to be received in the receiving groove (312) through a coupling means.

2. The apparatus as set forth in claim 1, wherein said coupling means comprises:
a guiding protrusion (313) which protrudes from a portion of the inner side of the receiving groove (312), and
a seat (429) which is formed at a portion of the bottom of the head (421) to lock the guiding protrusion (313).

3. The apparatus as set forth in claim 2, wherein said coupling means further comprises a locking projection (428) protruding from a portion of the bottom of the head (421) to prevent the guiding protrusion (313) from being separated from the seat (429).

4. The apparatus as set forth in claim 2, wherein the coupling means further comprises a guiding means for guiding the guiding protrusion (313) to be locked into the seat (429) and the guiding means comprises a fixing groove (315) formed at a side portion of the receiving groove (312) and a fixing protrusion (423) protruding from a side portion of the head (421) so as to fit into the fixing groove (315).

5. The apparatus as set forth in claim 4, wherein the guiding means further comprises a guide slope (427) formed at a predetermined angle on a portion of the bottom of the head (421) to facilitate the guiding protrusion (313) to be slid into the seat (429).

6. The apparatus as set forth in claim 3, wherein the coupling means further comprises a guiding means for guiding the guiding protrusion (313) to be locked into the seat (429) and the guiding means comprises a fixing groove (315) formed at a side portion of the receiving groove (312) and a fixing protrusion (423) protruding from a side portion of the head (421) so as to fit into the fixing groove (315).

7. The apparatus as set forth in claim 6, wherein the guiding means further comprises a guide slope (427) formed at a predetermined angle on a portion of the bottom of the head (421) to facilitate the guiding protrusion (313) to be slid into the seat (429).

8. The apparatus as set forth in claim 1, further comprising a needle assembly (100) configured to be inserted into the bone to form a path from the skin to the bone.

9. The apparatus as set forth in claim 8, wherein the needle assembly (100) comprises:
a pipe assembly (110) comprising a pipe (130) and a handle (111) connected to an end of the pipe (130) and having a receiving groove (112); and
a steel wire assembly (120) comprising a steel wire (122) that can be inserted into the pipe (130) of the pipe assembly (110) and a head (121) that is connected to an end of the steel wire (122) and is configured to be received in the receiving groove (112) through a coupling means.

10. The apparatus as set forth in claim 9, wherein the coupling means comprises:
a guiding protrusion (113) protruding from a portion of the inner side of the receiving groove (112); and
a seat (129) formed at a portion of the bottom of the head (121) to lock the guiding protrusion (113).

11. The apparatus as set forth in claim 10, wherein the coupling means further comprises a locking projection (128) protruding from a portion of the bottom of the head (121) to prevent the guiding protrusion (113) from being separated from the seat (129).

12. The apparatus as set forth in claim 10, wherein the coupling means further comprises a guiding means for guiding the guiding protrusion (113) that is locked into the seat (129) and the guiding means comprises fixing groove (115) formed at a side portion of the receiving groove (112) and a fixing protrusion (123) protruding from a side portion of the head (121) so as to fit into the fixing groove (115).

13. The apparatus as set forth in claim 12, wherein the guiding means further comprises a guide slope (127) formed at a predetermined angle on a portion of the bottom of the head (121) to facilitate the guiding protrusion (113) to be slid into the seat (129).

14. The apparatus as set forth in claim 11, wherein the coupling means further comprises a guiding means for guiding the guiding protrusion (113) that is locked into the seat (129) and the guiding means comprises fixing groove (115) formed at a side portion of the receiving groove (112) and a fixing protrusion (123) protruding from a side portion of the head (121) so as to fit into the fixing groove (115).

15. The apparatus as set forth in claim 14, wherein the guiding means further comprises a guide slope (127) formed at a predetermined angle on a portion of the bottom of the head (121) to facilitate the guiding protrusion (113) to be slid into the seat (129).

16. The apparatus as set forth in claim 1, further comprising a guiding instrument (200) that is formed of a wire pin and acts as a guide to the bone.

17. The apparatus as set forth in claim 1, further comprising a space forming instrument for forming inside the bone a space for a cavity-forming instrument to be inserted into.

18. The apparatus as set forth in claim 17, wherein the space forming instrument is a spacer assembly (500) which comprises:
a steel wire (522) having a threaded portion (520) at an end thereof and being able to be inserted into the pipe (330) of the cannula (300); and
a handle (511) connected to the other end of the steel wire (522).

19. The apparatus as set forth in claim 18, wherein the threaded portion (520) is formed of a double to fourfold-thread with a phase difference of 90° to 180° such that a distance that the thread moves forward in one turn thereof is equal to two to four times the axial distance between any point on a thread and a corresponding point on the next thread.

20. The apparatus as set forth in claim 18, wherein the threaded portion (520) is formed of a triple-thread with a phase difference of 120° such that a distance that the thread moves forward in one turn thereof is equal to three times the axial distance between any point on a thread and a corresponding point on the next thread.

21. The apparatus as set forth in claim 1, further comprising:
a filler assembly (600) configured to accommodate a bone-filling material therein; and
a pusher assembly (700) for pushing the bone-filling material inserted in the filler assembly 600.

22. The apparatus as set forth in claim 8, further comprising a guiding instrument (200) that is formed of a wire pin and acts as a guide to the bone.

23. The apparatus as set forth in claim 8, further comprising a space forming instrument for forming inside the bone a space for a cavity-forming instrument to be inserted into.

24. The apparatus as set forth in claim 23, wherein the space forming instrument is a spacer assembly (500) which comprises:
a steel wire (522) having a threaded portion (520) at an end thereof and being able to be inserted into the pipe (330) of the cannula (300); and
a handle (511) connected to the other end of the steel wire (522).
